(19) United States
(12) Patent Application Publication
(10) Pub. No.: US 2009/0126724 A1
(43) Pub. Date: May 21, 2009

(54) ONE-WAY VALVE

(75) Inventors: Dennis Robert Thiele, Darlington (AU); Paul Michael Watt, Mt. Claremont (AU)

Correspondence Address: DAVIS WRIGHT TREMAINE, LLP/Seattle, 1201 Third Avenue, Suite 2200, SEATTLE, WA, 98101-3045, US

(73) Assignee: INFAMED LIMITED, Nedlands, WA (AU)

(21) Appl. No.: 11/813,758

(22) PCT Filed: Jan. 12, 2006
(86) PCT No.: PCT/AU06/00029

§ 371 Date: Jan. 25, 2008

(30) Foreign Application Priority Data
Jan. 12, 2005 (AU) 2005900108

(51) Int. Cl. A61M 15/00; F16K 7/17; A61M 16/20; F16T 1/14

(52) U.S. Cl. 128/200.23; 251/331

(57) ABSTRACT

A one-way valve (10) is disclosed which comprises a body portion (12) defining a passage through which fluid is able to pass during use, a support member (22) disposed in the passage, a seat member (28) disposed in the passage and provided with at least one aperture (32), a shaft (24) extending between the support member (22) and the seat member (28) and a membrane (26) disposed on the shaft (24) such that the membrane (26) is rotatable relative to the shaft (24). The seat member (28) cooperates with the membrane (26) such that a first predetermined pressure differential across the membrane (26) causes the membrane to cover the at least one aperture (32) and thereby prevent fluid flow past the seat member (28), and such that a second predetermined pressure differential of opposite polarity to the first predetermined pressure differential causes the membrane (26) to not cover the at least one aperture (32) and thereby permit fluid flow past the seat member (28).

### FIELD OF THE INVENTION

The present invention relates to a one-way valve.

### BACKGROUND OF THE INVENTION

It is known to provide a one-way valve which includes a seat member provided with apertures and a membrane which is arranged so that at least part of the membrane moves away from or presses against the seat member to open or close the valve depending on whether a positive or negative pressure differential exists across the membrane.

However, such one-way valves often suffer from reliability and/or safety problems.

### SUMMARY OF THE INVENTION

In accordance with a first aspect of the present invention, there is provided a one-way valve comprising:
a body portion defining a passage through which fluid is able to pass during use;
a support member disposed in the passage;
a seat member disposed in the passage and provided with at least one aperture;
a shaft extending between the support member and the seat member; and
a membrane disposed on the shaft such that the membrane is rotatable relative to the shaft;
the seat member cooperating with the membrane such that a first predetermined pressure differential across the membrane causes the membrane to cover the at least one aperture and thereby prevent fluid flow past the seat member, and such that a second predetermined pressure differential of opposite polarity to the first predetermined pressure differential causes the membrane to not cover the at least one aperture and thereby permit fluid flow through the seat member and past the membrane and the support member.

In one arrangement, the seat member is provided with at least one upstanding portion, the at least one upstanding portion cooperating with the membrane so as to minimise contact surface area between the membrane and the seat member when the pressure differential across the membrane is substantially zero to thereby restrict sticking of the membrane to the seat member during use.

The support member is arranged such that a third predetermined pressure differential greater than the second predetermined pressure differential causes the membrane to deflect around the support member during use.

In one embodiment, the support member has a cross sectional profile which is curved so as to encourage predictable deflection of the membrane around the support member during use.

In one arrangement, the support member is substantially elongate.

The support member may be formed integrally with the body portion so as to avoid risk of dislodgement of the support member from the body portion during use.

In one arrangement, the seat member includes a plurality of vanes which define a plurality of apertures.

In one arrangement, the seat member is formed integrally with the body portion.

The seat member may be fixed to the body portion by ultrasonic welding, by spin welding, or by solvent bonding.

In addition or alternatively, the seat member and the body portion may be arranged such that the seat member and the body portion form an interference fit or a snap fit type connection.

In one embodiment, the at least one upstanding portion comprises a plurality of nodules or an upstanding ring member.

The membrane may be disposed such that the membrane is rotatable and reciprocably movable relative to the shaft.

The one-way valve may be incorporated into an inhaler suitable for dispensing medication to sufferers of a medical condition such as asthma, into anesthetic equipment, or into any other apparatus requiring a one-way valve.

### BRIEF DESCRIPTION OF THE DRAWINGS

The present invention will now be described, by way of example only, with reference to the accompanying drawings, in which:

FIG. 1 is a diagrammatic perspective view of a Y-piece including a one-way valve in accordance with an embodiment of the present invention;

FIG. 2 is a diagrammatic exploded perspective view of the Y-piece shown in FIG. 1;

FIG. 3 is a diagrammatic cross-sectional view of the Y-piece shown in FIG. 1;

FIG. 4 is a diagrammatic perspective view of the Y-piece shown in FIG. 1 with a seat member and a membrane of the one-way valve removed;

FIG. 5 is a diagrammatic perspective view of a seat member of the one-way valve incorporated into the Y-piece shown in FIG. 1;

FIG. 6 is a diagrammatic perspective view of a Y-piece including a one-way valve in accordance with an alternative embodiment of the present invention, with a seat member and a membrane removed; and

FIG. 7 is a diagrammatic perspective view of a seat member of the one-way valve incorporated into the Y-piece shown in FIG. 6.

### DESCRIPTION OF AN EMBODIMENT OF THE PRESENT INVENTION

The following embodiments of the invention will be described in relation to a Y-piece, in particular for use in an inhaler for delivering medication to children. However, it will be understood that the invention is not restricted to this application and the invention is applicable to any device having a one-way valve, including anesthetic apparatus.

Referring to the drawings, there is shown a Y-piece 10 including a one-way valve which in this example is for use in an inhaler of the type useable to dispense medication to sufferers of asthma, in particular children.

In this example, the one-way valve incorporated into the Y-piece 10 includes a body portion 12 of generally cylindrical configuration, a support member 22, a shaft 24, a membrane 26 and a seat member 28.

The body portion 12 defines a passage through which fluid is able to pass during use, and the body portion 12 includes a first end 14 and a second opposite end 16.

Integral with or connected to the body portion 12 is a side arm 18, the side arm 18 having a free third end 20 remote from the body portion 12. The body portion 12 and the side arm 18 define a Y-piece which in this example is arranged so as to permit fluid flow from the second end 16 to the first end 14 when the pressure adjacent the first end 14 is less than the pressure adjacent the second end 16. The Y-piece is also arranged so as to restrict fluid flow from the first end 14 to the second end 16 when the pressure adjacent the first end 14 and the third end 20 is greater than the pressure adjacent the second end 16. In this way, it is possible to provide a reservoir of medication connected to the second end 16 for delivery to a patient when the patient inhales, and to restrict air flow from the patient to the medication reservoir when the patient exhales.

In this example, the support member 22 is of generally elongate configuration, the support member 22 extending across the passage defined by the body portion 12. The support member 22 is integrally formed with the body portion 12 so that when the one-way valve is used as part of an inhaler with a mouthpiece connected to the first end 14 and a medication reservoir connected to the second end 16, there is no risk of dislodgement of the support member 22 and thereby injury to a patient as the patient inhales medication during use.

The support member 22 is integrally formed with the shaft 24 and is disposed generally centrally of the support member 22, the shaft 24 receiving a membrane 26 formed of flexible material such as silicone. The membrane 26 has an aperture 27 and is disposed on the shaft 24 such that the membrane is rotatable and reciprocably movable relative to the shaft 24. Since in this example the transverse cross-sectional shape of the passage of the body portion 12 is generally circular, the shape of the membrane 26 is also generally circular.

It will be understood that since the membrane 26 is disposed on the shaft 24 which is integral with the support member 22, there is also no risk of dislodgement of the membrane 26 and thereby injury to a patient as the patient inhales during use.

The support member has a cross-sectional profile which is curved and preferably semi-circular so that during use when the membrane 26 deflects around the support member, the deflection occurs in a predictable way.

The support member 22 and the seat member 28 define a gap of predetermined spacing for receiving the membrane 26.

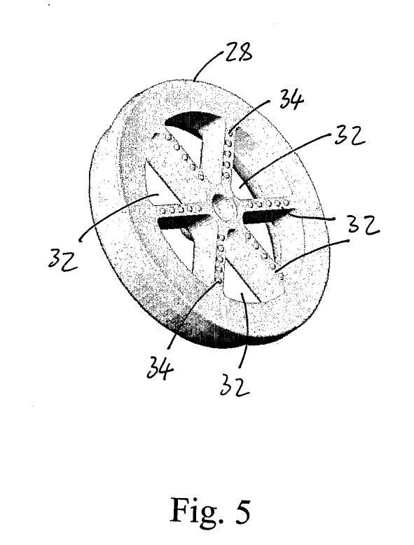

In this example, the seat member 28 is provided with a plurality of vanes 30 defining a plurality of apertures 32 therebetween. As shown more particularly in FIG. 5, the seat member 28 also includes at least one upstanding portion, in this example a plurality of nodules 34. However, it will be understood that other upstanding portion configurations are possible, the important aspect being that the at least one upstanding portion functions to minimise the contact surface area between the seat member and the membrane 26 when the pressure differential across the membrane is substantially equal to zero. For example, the at least one upstanding portion may be in the form of an upstanding ring.

In this example, during manufacture, the body portion 12, the support member 22 and the shaft 24 are formed integrally with each other, the membrane 26 is disposed on the shaft 24, and the seat member 28 is fixed relative to the body portion 12 using any suitable fixing means, for example using ultrasonic welding, by spin welding, or by solvent bonding.

As an alternative, the seat member and the body portion may be arranged such that the seat member and the body portion form an interference fit or a snap fit type connection.

As a further alternative, the body portion 12 and the seat member 28 may be formed integrally with each other, and the support member 22 fixed relative to the body portion 12 using any suitable fixing means.

It will be understood that when the pressure adjacent the first and third ends 14, 20 is lower than the pressure adjacent the second end 16, at least part of the membrane moves away from the seat member 28 to thereby open the one way valve and permit fluid flow from the second end 16 to the first end 14, and that when the pressure adjacent the first and third ends 14, 20 is lower than the pressure adjacent the second end 16 by a sufficient amount, the membrane 26 will be caused to deflect around the support member 22. Importantly, since the profile of the support member is curved, the deflection will occur in a predetermined and predictable way. This is significant since deflection predictability ensures that the pressure differential required to cause deflection of the membrane 26 around the support member 22 is always substantially the same and a user does not perceive any difference between successive inhalations.

It will also be understood that since the membrane 26 is retained by the shaft 24 and the support member 22 whilst allowing the membrane 26 to rotate and move reciprocally relative to the shaft, the likelihood of failure of the membrane 26 during use is reduced because the membrane 26 is not constructed to deform at the same location with each opening of the valve.

In addition, with the present example, it will also be understood that since the membrane 26 is deflected about an elongate support member 22, minimal force will be exerted by the membrane 26 to resist deflection. This is particularly significant for child inhalers wherein it is desirable to minimise the patient effort required to effect inhalation of medication.

It will also be understood that when the pressure adjacent the first end 14 and the third end 20 is greater than the pressure adjacent the second end 16, for example during exhalation, the membrane 26 will be caused to press against the seat member 28 and thereby prevent passage of fluid through the apertures 32.

It will also be understood that when the pressure adjacent the first end 14 is substantially equal to the pressure adjacent the second end 16, the membrane 26 will contact only a small proportion of the surface area of the seat member 28 provided by the nodules 34. As a consequence, the likelihood of the membrane 26 sticking to the seat member 28 during use is significantly reduced compared to one-way valves known hitherto. A further consequence is that the valve is normally open, thereby ensuring that minimal effort will be required to effect inhalation of medication.

Furthermore, it will be understood that since the support member 22 and the shaft 24 are formed integrally with the body portion 12, the method of fabrication of the one-way valve is relatively simple as fabrication only requires disposal of the membrane 26 and the seat member on the shaft 24 and fixing of the seat member 28 relative to the body portion 12.

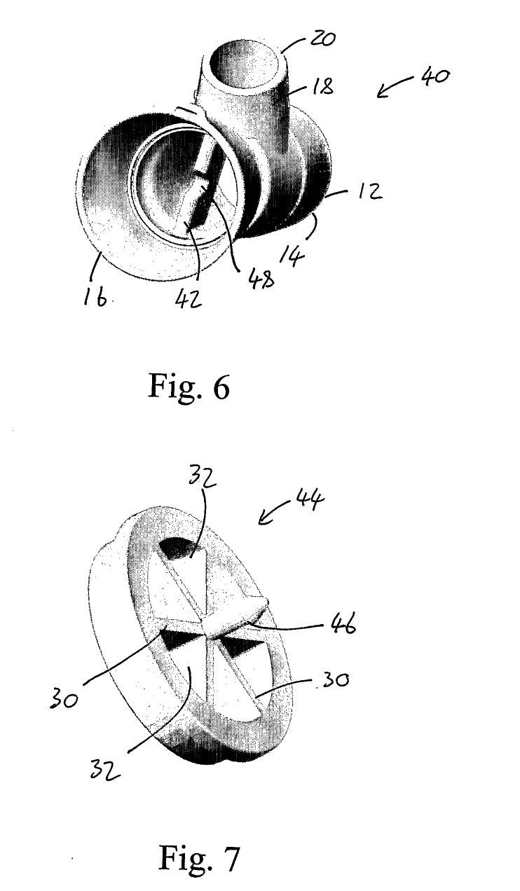

An alternative embodiment of a Y-piece 40 incorporating a one-way valve is shown in FIGS. 6 and 7. Like and similar features are indicated with like reference numerals.

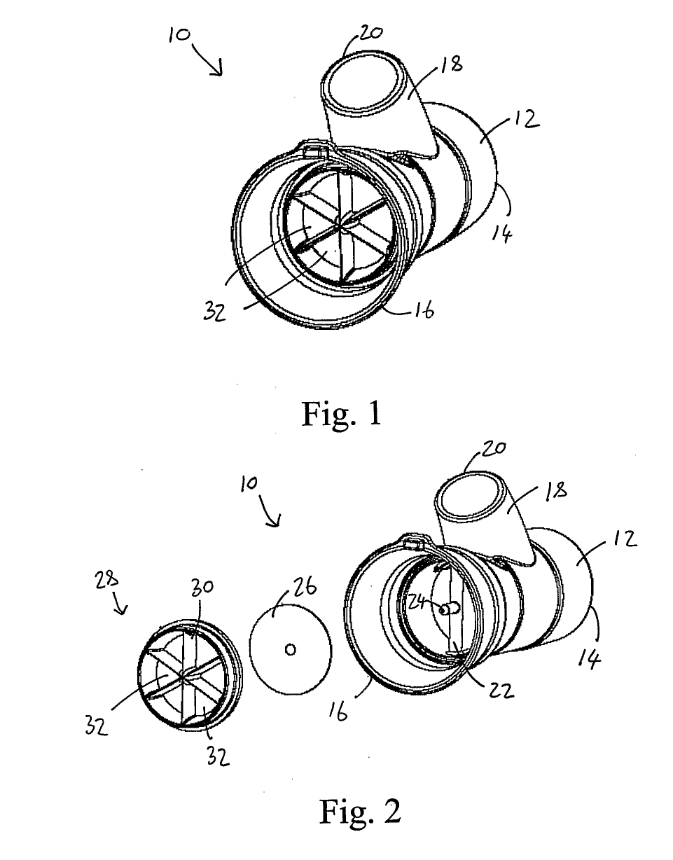

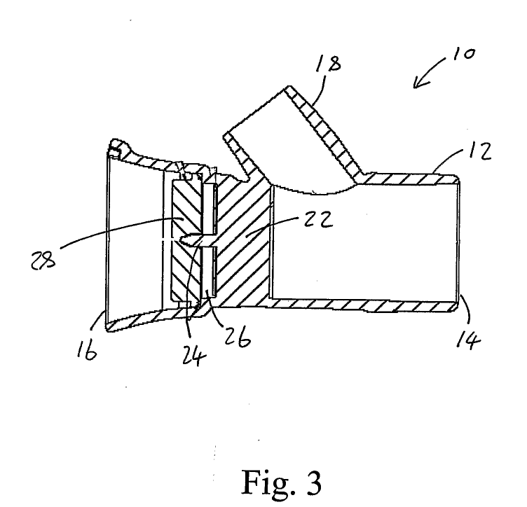

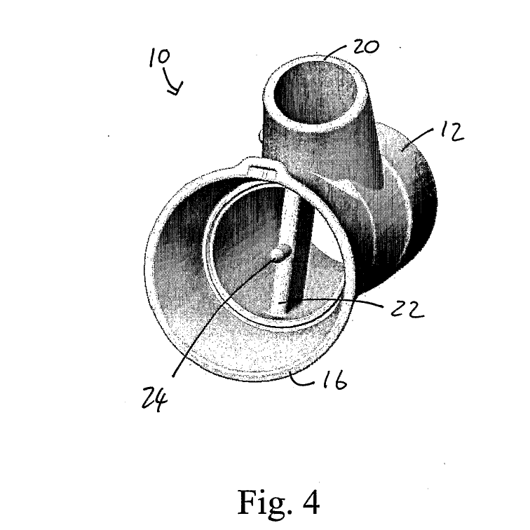

The alternative one-way valve includes an alternative elongate support member 42 which is formed integrally with the body portion 12, and an alternative seat member 44 formed integrally with a shaft 46, as shown more particularly in FIG. 7. As with the embodiment shown in FIGS. 1 to 5, a membrane (not shown) is disposed between the support member 42 and the seat member 44 on the shaft 46.

The embodiment shown in FIGS. 6 and 7 differs from the embodiment shown in FIGS. 1 to 5 in that a shaft 46 is formed on the seat member 44 instead of on the support member 42. A recess 48 is also provided on the support member 42 for receiving the shaft 46 when the seat member 44 is fixed relative to the body portion 12.

Although the seat member 44 shown in FIG. 7 does not include nodules, it will be understood that nodules may be included to assist in minimising sticking of the membrane 26 to the seat member 44 during use and to ensure that the valve is normally open when the pressure differential across the membrane is approximately zero.

While the above embodiments are described in relation to a one-way valve for use in an inhaler, it will be appreciated that the invention is applicable to any application requiring a reliable and/or safe one-way valve.

Modifications and variations as would be apparent to a skilled addressee are deemed to be within the scope of the present invention.

1. A one-way valve comprising:
a body portion defining a passage through which fluid is able to pass during use;
a support member disposed in the passage;
a seat member disposed in the passage and provided with at least one aperture;
a shaft extending between the support member and the seat member; and
a membrane disposed on the shaft such that the membrane is rotatable relative to the shaft;
the seat member cooperating with the membrane such that a first predetermined pressure differential across the membrane causes the membrane to cover the at least one aperture and thereby prevent fluid flow past the seat member, and such that a second predetermined pressure differential of opposite polarity to the first predetermined pressure differential causes the membrane to not cover the at least one aperture and thereby permit fluid flow past the seat member.

2. A one-way valve as claimed in claim 1, wherein the seat member is provided with at least one upstanding portion, the at least one upstanding portion cooperating with the membrane so as to minimise contact surface area between the membrane and the seat member when the pressure differential across the membrane is substantially zero to thereby restrict sticking of the membrane to the seat member during use.

3. A one-way valve as claimed in claim 2, wherein the at least one upstanding portion comprises a plurality of nodules.

4. A one-way valve as claimed in claim 2, wherein the at least one upstanding portion comprises an upstanding ring member.

5. A one-way valve as claimed in claim 1, wherein the support member is arranged such that a third predetermined pressure differential greater than the second predetermined pressure differential causes the membrane to deflect around the elongate support member during use.

6. A one-way valve as claimed in claim 5, wherein the support member has a cross sectional profile which is curved so as to encourage predictable deflection of the membrane around the support member during use.

7. A one-way valve as claimed in claim 5, wherein the support member is substantially elongate.

8. A one-way valve as claimed in claim 1, wherein the support member is formed integrally with the body portion.

9. A one-way valve as claimed in claim 1, wherein the seat member includes a plurality of vanes which define a plurality of apertures.

10. A one-way valve as claimed in claim 1, wherein the seat member is formed integrally with the body portion.

11. A one-way valve as claimed in claim 1, wherein the membrane is disposed on the shaft such that the membrane is rotatable and reciprocably movable relative to the shaft

12. An inhaler comprising a one-way valve as claimed in claim 1.

13. An anaesthetic apparatus comprising a one-way valve as claimed in claim 1.
